(19) United States
(12) Patent Application Publication
(10) Pub. No.: US 2019/0183114 A1
(43) Pub. Date: Jun. 20, 2019

(54) METHODS AND COMPOSITIONS FOR THE PRESERVATIONOF ORGANS

(71) Applicants: INSERM (Institut National de la Sante et de la Recherche Medicale), Paris (FR); Universite de Poitiers, Poitiers (FR); Centre Hospitalier Universitaire de Poitiers, Poitiers (FR)

(72) Inventors: Thierry HAUET, Poitiers cedex (FR); Sylvain LE PAPE, Poitiers cedex (FR); Raphael THUILLIER, II, Poitiers cedex (FR)

(21) Appl. No.: 16/322,159

(22) PCT Filed: Aug. 4, 2017
(86) PCT No.: PCT/EP2017/069774

§ 371 Date: Jan. 31, 2019

(30) Foreign Application Priority Data
Aug. 5, 2016 (EP) 16306017.1

(51) Int. Cl. A01N 1/02; C12N 15/113

(52) U.S. Cl. CPC A01N 1/0278; A01N 1/0242; C12N 15/113; A01N 1/0226

(57) ABSTRACT

The present invention relates to methods and compositions for the preservation of organs. The inventors showed that therapeutic intervention during cold ischemia could significantly alter outcome, suggesting active mechanisms taking place. More particularly, the inventors demonstrated that Unfolded Protein Response could be a critical pathway underlying the relationship between cold ischemia time and graft outcome, highlighting the potential for UPR-based therapeutics to improve transplantation efficiency. In particular, the present invention relates to an activator of PERK-ATF4 pathway, an inhibitor of the ATF6 pathway and an inhibitor of the RNase activity of IRE1α for use in the maintain of organ viability during cold ischemia.

### FIELD OF THE INVENTION

The present invention relates to methods and compositions for the preservation of organs.

### BACKGROUND OF THE INVENTION

Transplantation is currently the most efficient therapy for end stage organ diseases. There is an increasing demand for organs, however donation rates are low. In France, the 2014 kidney waiting list registered 20311 requests, of which only 5357 were fulfilled. Such shortage is a worldwide trend. In recent years, efforts have been made to extend deceased donor inclusion parameters, creating an ‘extended criteria donor’ category (Rosengard B R, Feng S, Alfrey E J et al. Report of the Crystal City meeting to maximize the use of organs recovered from the cadaver donor. Am. J. Transplant. Off. J. Am. Soc. Transplant. Am. Soc. Transpl. Surg. 2002; 2: 701-711). However, even such radical change could not reduce the shortage and the gap between organ demand and availability is ever increasing.

New types of donors will need to be sought, which will likely impact organ quality and outcome. Indeed, extended criteria donors are characterized by poor organ quality, with increased sensitivity to ischemia reperfusion injury (IRI) (Meier-Kriesche H-U, Schold J D, Srinivas T R et al. Lack of improvement in renal allograft survival despite a marked decrease in acute rejection rates over the most recent era. Am. J. Transplant. Off. J. Am. Soc. Transplant. Am. Soc. Transpl. Surg. 2004; 4: 378-383) that directly impacts on post-transplant complications (Singh S K, Kim S J. Does expanded criteria donor status modify the outcomes of kidney transplantation from donors after cardiac death? Am. J. Transplant. Off. J. Am. Soc. Transplant. Am. Soc. Transpl. Surg. 2013; 13: 329-336). Ischemia reperfusion (IR) (Favreau F, Giraud S, Bon D et al. Ischemia reperfusion control: the key of kidney graft outcome. Médecine Sci. MS 2013; 29: 183-188.) is unavoidable in transplantation: ischemia starts when the organ is cut from circulating blood and will last through transport and implantation until anastomoses are completed. Reperfusion starts with the restoration of blood flow, and is characterized by an activation of pathological mechanisms, among which oxidative stress and inflammation. These mechanisms, which are still not completely characterized, are directly correlated with outcome (Russo M J, Chen J M, Sorabella R A et al. The effect of ischemic time on survival after heart transplantation varies by donor age: An analysis of the United Network for Organ Sharing database. J. Thorac. Cardiovasc. Surg. 2007; 133: 554-559), (Patel N D, Weiss E S, Nwakanma L U et al. Impact of Donor-to-Recipient Weight Ratio on Survival After Heart Transplantation Analysis of the United Network for Organ Sharing Database. Circulation 2008; 118: S83-S88).

It is demonstrated that the intensity of IR lesions is correlated to cold ischemia time (CIT), where each hour increases the risk of complications (Van der Vliet J A, Warlé M C. The need to reduce cold ischemia time in kidney transplantation. Curr. Opin. Organ Transplant. 2013; 18: 174-178), (Debout A, Foucher Y, Trébern-Launay K et al. Each additional hour of cold ischemia time significantly increases the risk of graft failure and mortality following renal transplantation. Kidney Int. 2015; 87: 343-349), (Ponticelli C E. The impact of cold ischemia time on renal transplant outcome. Kidney Int. 2015; 87: 272-275). However, the underlying mechanisms remain to be defined, which slows the development of adapted therapeutics.

### SUMMARY OF THE INVENTION

The present invention relates to methods and compositions for the preservation of organs. In particular, the present invention is defined by the claims.

### DETAILED DESCRIPTION OF THE INVENTION

Surprisingly, while common dogma assimilates cold ischemia to a stasis characterized by a hypothermia-induced slowing of metabolism, the inventors have shown that therapeutic intervention during cold ischemia could significantly alter outcome, suggesting active mechanisms taking place. More particularly, the inventors demonstrate that UPR (Unfolded Protein Response) could be a critical pathway underlying the relationship between CIT and graft outcome, highlighting the potential for UPR-based therapeutics to improve transplantation efficiency.

### Methods of the Present Invention

A first aspect of the present invention relates to a method of maintaining organ viability during cold ischemia comprising perfusing the organ with a preservation solution wherein:

an effective amount of an activator of PERK-ATF4 pathway is added in the first six hours of cold ischemia time;

and if the expected cold ischemia time exceeds 8 hours, adding to said preservation solution:

an effective amount of an inhibitor of the ATF6 pathway in the first twelve hours of cold ischemia time and/or,

an effective amount of an inhibitor of the RNase activity of IRE1α at least one hour before reperfusion of the organ.

As used herein, the term “organ” refers to a part or structure of the body, which is adapted for a special function or functions. In a particular embodiment, the organ is the lungs, the liver, the kidneys, the heart, the pancreas and the bowel, including the stomach and intestines.

As used herein, the term “organ viability” refers to the capacity of the organ to resume an acceptable level of function upon transplantation due to the good quality of its preservation.

As used herein, the term “cold ischemia time” (“CIT”) has its general meaning in the art and refers to the time which extends from the initiation of cold preservation of the recovered organ to restoration of warm circulation after transplantation. There is variability by accepting surgeon/center and by donor and recipient characteristics. Intuitively, shorter CIT is better. For kidney transplantation, the CIT should be inferior to 24 hours; for pancreas transplantation, the CIT should be inferior to 18 hours and for liver transplantation, the CIT should be inferior to 8 hours (Bernat J L, D'Alessandro A M, Port F K, Bleck T P, Heard S O, Medina J, et al. Report of a National Conference on Donation after cardiac death. Am J Transplant. 2006; 6:281-91).

As used herein, the term “expected cold ischemia time” refers to the duration of cold ischemia reasonably anticipated by the surgeon/health professional.

As used herein, the term “reperfusion” refers to the restauration of blood flow to a previously ischemic organ.

As used herein, the term “pathway” relates to a set of system components involved in two or more sequential molecular interactions that result in the production of a product or activity. A pathway can produce a variety of products or activities that can include, for example, intermolecular interactions, changes in expression of a nucleic acid or polypeptide, the formation or dissociation of a complex between two or more molecules, accumulation or destruction of a metabolic product, activation or deactivation of an enzyme or binding activity. Thus, the term “pathway” includes a variety of pathway types, such as, for example, a biochemical pathway, a gene expression pathway, and a regulatory pathway. Similarly, a pathway can include a combination of these representative pathway types.

As used herein the term “PERK” has its general meaning in the art and refers to the eukaryotic translation initiation factor 2-alpha kinase 3 (Shi Y, et al. (1998) “Identification and characterization of pancreatic eukaryotic initiation factor 2 alpha-subunit kinase, PEK, involved in translational control”. Mol Cell Biol. 18(12):7499-509).

As used herein the term “ATF4” has its general meaning in the art and refers to the Activating transcription factor 4 (tax-responsive enhancer element B67) (Tsujimoto A et al. (1991). “Isolation of cDNAs for DNA-binding proteins which specifically bind to a tax-responsive enhancer element in the long terminal repeat of human T-cell leukemia virus type I”. Journal of Virology 65 (3): 1420-6. Karpinski B A et al. (1992). “Molecular cloning of human CREB-2: an ATF/CREB transcription factor that can negatively regulate transcription from the cAMP response element”. Proceedings of the National Academy of Sciences of the United States of America 89 (11): 4820-4.)

As used herein, the term “ATF6” has its general meaning in the art and refers to the activating transcription factor 6. ATF6 is a factor that activates target genes for the unfolded protein response (UPR) during endoplasmic reticulum (ER) stress (Haze K., Yoshida H., Yanagi H., Yura T., Mori K. Mammalian transcription factor ATF6 is synthesized as a transmembrane protein and activated by proteolysis in response to endoplasmic reticulum stress. Mol. Biol. Cell. 1999; 10:3787-3799. doi: 10.1091/mbc.10.11.3787).

As used herein the term “IRE1” has its general meaning in the art and refers to serine/threonine-protein kinase/endoribonuclease inositol-requiring enzyme 1 (Tirasophon W., Welihinda A. A. & Kaufman R. J. A stress response pathway from the endoplasmic reticulum to the nucleus requires a novel bifunctional protein kinase/endoribonuclease (Irelp) in mammalian cells. Genes Dev. 12, 1812-1824 (1998)).

As used herein, the term “RNase activity of IRE1” refers to the activity of the endoribonuclease domain of IRE1 which either degrades specific RNA to avoid their translation, an activity known as the RIDD (regulated IRE1-dependent decay of mRNA), or splices XBP1 (X-box-binding protein 1) mRNA to generate a new C-terminus, converting it into a potent unfolded-protein response transcriptional activator and triggering growth arrest and apoptosis.

As used herein, the term “activator of PERK-ATF4 pathway” refers to any compound which interacts with one or more molecule of the PERK-ATF4 pathway resulting in the activation of the pathway.

As used herein, the term “inhibitor of the ATF6 pathway” refers to any compound which interacts with one or more molecule of the inhibitor of the ATF6 pathway resulting in the inhibition of the pathway.

As used herein, the term “inhibitor of RNase activity of IRE1” refers to any compound which interacts with IRE1 resulting in the inhibition of its RNase activity.

As used herein, the terms “preservation solution” or “organ preservation solution” refer to an aqueous solution having a pH between 6.5 and 7.5, including salts, preferably chloride, sulfate, sodium, calcium, magnesium and potassium; sugars, preferably mannitol, raffinose, sucrose, glucose, fructose, lactobionate (which is a water resistant), or gluconate; antioxidants, for instance glutathione; active agents, for instance xanthine oxidase inhibitors such as allopurinol, lactates, amino acids such as histidine, glutamic acid (or glutamate), tryptophan; and optionally colloids such as hydroxyethyl starch, polyethylene glycol or dextran.

In one embodiment of the invention, the organ preservation solution is selected from:
the solution from the University of Wisconsin (UW or ViaSpan®), which has an osmolality of 320 mOsmol/kg and a pH of 7.4, of the following formulation for one liter in water: potassium lactobionate: 100 mM, KOH: 100 mM, NaOH: 27 mM, KH2PO4: 25 mM, MgSO4: 5 mM, Raffinose: 30 mM, Adenosine: 5 mM, Glutathione: 3 mM, Allopurinol: 1 mM, Hydroxyethyl starch: 50 g/L,
IGL-1®, having an osmolality of 320 mOsm/kg and a pH of 7.4, of the following formulation, per liter in water: NaCL:125 mM, KH2PO4: 25 mM, MgSO4: 5 mM, Raffinose: 30 mM, potassium lactobionate: 100 mM, Glutathione: 3 mM, Allopurinol: 1 mM, Adenosine: 5 mM, Polyethylene glycol (molecular weight: 35 kDa): 1 g/L,
Celsior®, having an osmolality of 320 mOsm/kg and a pH of 7.3, of the following formulation per liter in water: Glutathione: 3 mM, Mannitol: 60 mM, lactobionic acid: 80 mM, Glutamic acid: 20 mM, NaOH: 100 mM, calcium chloride dehydrate: 0.25 mM, MgSO4: 1.2 mM, KCl: 15 mM, magnesium chloride hexahydrate: 13 mM, Histidine 30 mM,
BMPS Belzer® or Belzer solution infusion machine or KPS1, especially comprising 100 mEq/L of sodium, 25 mEq/L potassium, pH 7.4 at ambient temperature, and having an osmolarity of 300 mOsm/L,
Custodiol® HTK solution having the following formulation per liter in water, the pH of 15 being 7.20 at room temperature, and the osmolality was 310 mOsm/kg: NaCl: 18.0 mM, KCl: 15.0 mM, KH2PO4: 9 mM, 2-ketoglutarate hydrogenated potassium: 1.0 mM, hexahydrate magnesium chloride: 4.0 mM; histidine, HCl, H2O: 18.0 mM, histidine: 198.0 mM, Tryptophan: 2.0 mM, Mannitol: 30.0 mM, calcium chloride dihydrate: 0.015 mM
Soltran®, having an osmolality of 486 mOsm/kg and a pH of 7.1 and the following formulation per liter in water: Sodium: 84 mM, Potassium: 80 mM, Magnesium: 41 mM, Sulfate: 41 mM, Mannitol 33.8 g/1, Citrate: 54 mM, Glucose: 194 mM,
Perfadex®, having an osmolarity of 295 mOsmol/L and the following formulation in water: 50 g/L of Dextran 40 (molecular weight: 40,000), Na+138 mM, K+6 mM, Mg2+: 0.8 mM, Cl−142 mM, SO42 0.8 mM, (+H2PO4-HPO42-): 0.8 mM, glucose 5 mM,
Ringer Lactate®, of the following formulation, in water, the pH being between 6.0 and 7.5 at ambient temperature, and having an osmolarity of 276.8 mOsmol/L: Na+130 mM, K+5.4 mM, Ca2+: 1.8 mM, Cl−: 111 mM, Lactate: 27.7 mM,
Plegisol®, of the following formulation, in water: KCl: 1.193 g/1, MgCl2, H2O: 3.253 g/L, NaCl: 6.43 g/L, CaCl₂): 0.176 g/1,
Solution Hospital Edouard Henriot, of the following formulation in water, the pH being equal to 7.4 at ambient temperature, and having an osmolarity of 320 mOsmol/L: KOH: 25 mM, NaOH: 125 mm, KH2PO4: 25 mM, MgCl2: 5 mM, MgSO4: 5 mM, Raffinose: 30 mM, lactobionate: 100 mM, Glutathione: 3 mM, Allopurinol: 1 mM, Adenosine: 5 mM, Hydroxyethyl starch 50 g/L,
And Steen® solution comprising human serum albumin, dextran and extracellular electrolyte with a low concentration of potassium.

All these organ preservation solutions are commercial products.

In a particular embodiment, the preservation solution according to the invention is the solution from the University of Wisconsin (UW or Viaspan®).

In one embodiment, the activator of PERK-ATF4 pathway is added immediately after cold ischemia starts.

In one embodiment, the inhibitor of the ATF6 pathway is added in the first six hours of cold ischemia time.

In one embodiment, the inhibitor of the ATF6 pathway is added in the first hour of cold ischemia time.

The Activator of PERK-ATF4 Pathway

In a particular embodiment, the activator of PERK-ATF4 pathway is an agent that inhibits the dephosphorylation of phosphorylated eIF2a.

As used herein the term “eIF2a” has its general meaning in the art and refers to the eukaryotic translation initiation factor 2A that is a 65-kD protein that catalyzes the formation of puromycin-sensitive 80S preinitiation complexes (Zoll W L et al. (2002). “Characterization of mammalian eIF2A and identification of the yeast homolog”. J Biol Chem 277 (40): 37079-87; Merrick W C (1992). “Mechanism and regulation of eukaryotic protein synthesis”. Microbiol Rev 56 (2): 291-315).

In a particular embodiment, the activator of PERK-ATF4 pathway is salubrinal (3-phenyl-N-[2,2,2-trichloro-1-[[(8-quinolinylamino)thioxomethyl]amino]ethyl]-2-propenamide), which is an agent that inhibits the dephosphorylation of phosphorylated eIF2a. (Boyce et al (2005) “A selective inhibitor of eIF2alpha dephosphorylation protects cells from ER stress”. Science 307 935. Long et al (2005) “Structure-activity relationship studies of salubrinal lead to its active biotinylated derivative”. Bioorg. Med. Chem. Lett. 15 3849). Salubrinal is a cell-permeable, selective inhibitor of cellular phosphatase complexes that dephosphorylate eIF2a. Salubrinal is available from Alexis Biochemicals or Tocris Bioscience (Cat No. 2347), or other source as known to one of skill in the art.

In a particular embodiment, the activator of PERK-ATF4 pathway is guanabenz (2-(2,6-dichlorobenzylidene) hydrazinecarboximidamide), which is an agent that inhibits the dephosphorylation of phosphorylated eIF2a. Guanabenz is a small-molecule which bound to a regulatory subunit of protein phosphatase 1, PPP1R15A/GADD34, selectively disrupting the stress induced dephosphorylation of the a subunit of eIF2a (Tsaytler P, Harding H P, Ron D, Bertolotti “A. Selective inhibition of a regulatory subunit of protein phosphatase 1 restores proteostasis.”. Science. 2011 Apr. 1; 332(6025):91-4)

In a particular embodiment, the activator of PERK-ATF4 pathway is an inhibitor of GADD34 or PP1 gene expression, which is an agent that inhibits the dephosphorylation of phosphorylated eIF2.

In one embodiment, the activator of PERK-ATF4 pathway, as inhibitors of GADD34 or PP1 gene expression, for use in the present invention may be based on anti-sense oligonucleotide constructs. Anti-sense oligonucleotides, including anti-sense RNA molecules and anti-sense DNA molecules, would act to directly block the translation of GADD34 or PP1 mRNA by binding thereto and thus preventing protein translation or increasing mRNA degradation, thus decreasing the level of GADD34 or PP1, and thus activity, in a cell. For example, antisense oligonucleotides of at least about 15 bases and complementary to unique regions of the mRNA transcript sequence encoding GADD34 or PP1 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

In one embodiment, the activator of PERK-ATF4 pathway, as inhibitors of GADD34 or PP1 gene expression, is small inhibitory RNAs (siRNAs). GADD34 or PP1 gene expression can be reduced by transfecting a cell with a small double stranded RNA (dsRNA), or a vector or construct causing the production of a small double stranded RNA, such that GADD34 or PP1 gene expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836). All or part of the phosphodiester bonds of the siRNAs of the invention are advantageously protected. This protection is generally implemented via the chemical route using methods that are known by art. The phosphodiester bonds can be protected, for example, by a thiol or amine functional group or by a phenyl group. The 5′- and/or 3′-ends of the siRNAs of the invention are also advantageously protected, for example, using the technique described above for protecting the phosphodiester bonds. The siRNA sequences advantageously comprise at least twelve contiguous dinucleotides or their derivatives.

shRNAs (short hairpin RNAs) can also function as inhibitors of expression for use in the present invention.

In one embodiment, the activator of PERK-ATF4 pathway, as inhibitors of GADD34 or PP1 gene expression, is a ribozyme. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of GADD34 or PP1 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable.

Both antisense oligonucleotides and ribozymes useful as inhibitors of expression can be prepared by known methods. These include techniques for chemical synthesis such as by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-0-methyl rather than phosphodiesterase linkages within the oligonucleotide backbone.

In a particular embodiment, the activator of PERK-ATF4 pathway is chosen from the examples of GADD34 inhibitor described in the patent application US20100016235.

The Inhibitor of the ATF6 Pathway

In a particular embodiment, the inhibitor of the ATF6 pathway is an inhibitor of Site-1-protease (S1P) and Site-2-protease (S2P). S1P and S2P are involved in the release of the transcription factor domain of ATF6.

In a particular embodiment, the inhibitor of the ATF6 pathway is AEBSF, which is an inhibitor of Site-1-protease and Site-2-protease.

As used herein, the term “AEBSF” has its general meaning in the art and refers to 4-(2-Aminoethyl) benzenesulfonyl fluoride hydrochloride, which is a water-soluble, irreversible serine protease inhibitor.

In some embodiment, the inhibitor of the ATF6 pathway is an inhibitor of expression. In particular, the inhibitor of the ATF6 pathway is an inhibitor of ATF6 gene expression.

In some embodiments, the inhibitor of the ATF6 pathway is an inhibitor of ATF6 gene expression, such as siRNA, antisense oligonucleotide or a ribozyme

In some embodiments, the inhibitor of the ATF6 pathway is anti-sense oligonucleotides.

Anti-sense oligonucleotides, including anti-sense RNA molecules and anti-sense DNA molecules, would act to directly block the translation of target gene mRNA by binding thereto and thus preventing protein translation or increasing mRNA degradation, thus decreasing the level of ATF6, and thus activity, in a cell. For example, antisense oligonucleotides complementary to unique regions of the mRNA transcript sequence encoding ATF6 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

In a particular embodiment, the inhibitor of the ATF6 pathway is a small inhibitory RNA.

Small inhibitory RNAs (siRNAs) can function as inhibitors of ATF6 gene expression for use in the present invention. ATF6 gene expression can be reduced by transfecting a cell with a small double stranded RNA (dsRNA), or a vector or construct causing the production of a small double stranded RNA, such that ATF6 gene expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

In a particular embodiment, the inhibitor of the ATF6 pathway is a ribozyme.

Ribozymes can also function as inhibitors of ATF6 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of ATF6 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

In one embodiment, the inhibitor of the ATF6 pathway is a 4-phenylbutyric acid analogue (Zhang H, Nakajima S, Kato H, Gu L, Yoshitomi T, Nagai K, et al. Selective, potent blockade of the IRE1 and ATF6 pathways by 4-phenylbutyric acid analogues. Br J Pharmacol. oct 2013; 170(4):822-34).

The Inhibitor of the RNase Activity of IRE1α

In a particular embodiment, the inhibitor of the RNase activity of IRE1α is STF083010.

As used herein, the term “STF083010” has its general meaning in the art and refers to N-[(2-Hydroxy-1-naphthalenyl)methylene]-2-thiophenesulfonamide.

In one embodiment, the inhibitor of the RNase activity of IRE1α is 4μ8c.

In one embodiment, the inhibitor of the RNase activity of IRE1α is Irestatin.

In one embodiment, the inhibitor of the RNase activity of IRE1α is MG132.

In one embodiment, the inhibitor of the RNase activity of IRE1α is 17-AAG.

In one embodiment, the inhibitor of the RNase activity of IRE1α is 1-NM-PP1.

In one embodiment, the inhibitor of the RNase activity of IRE1α is Lactacystin.

In one embodiment, the inhibitor of the RNase activity of IRE1α is a 4-phenylbutyric acid analogue (Zhang H, Nakajima S, Kato H, Gu L, Yoshitomi T, Nagai K, et al. Selective, potent blockade of the IRE1 and ATF6 pathways by 4-phenylbutyric acid analogues. Br J Pharmacol. oct 2013; 170(4):822-34).

### Screening Method

Another aspect of the present invention relates to a method for screening modulators of the Unfold Protein Response for the maintenance of organ viability comprising the steps of i) providing a plurality of test substances ii) determining whether the test substances are UPR modulators and iii) positively selecting the test substances that are UPR modulators.

In one embodiment, the method of screening modulators of the UPR is performed in vitro as follows:

Primary human aortic endothelial cells (HAEC, Gibco) are cultured on 1% gelatin (Sigma) coated flasks of 75 cm² in M200 medium supplemented with LSGS (Gibco), 8% fetal bovine serum (FBS), and 100 μg/mL penicillin and streptomycin in a humidified atmosphere at 21% O2, 5% CO2 and 37° C. Cells are split at a ratio of 1:4 every 5 days. Cells up to passage 5 are used. Induction of the UPR is performed by addition of Tunicamycin (2 μg/L), with or without the candidate molecules.

Action on the UPR branches is measured using Real Time Polymerase Chain Reaction: RNA is extracted from HAEC using the NucleoSpin RNA kit (Macherey-Nagel) containing a DNase treatment to remove potentially contaminating genomic DNA. RNA quality is verified by resolution on a 1.5% (wt/vol) agarose gel and measurement of A260 nm/A280 nm and A260 nm/A230 nm ratios using a NanoDrop™ 2000 (Thermo Scientific). A quantity of 1 μg of total RNA is reverse-transcribed with High Capacity cDNA Reverse Transcription kit (ABsystems). RT-qPCR is performed in triplicate with negative template controls, negative enzyme controls and the use of a calibrator to limit inter-run variations as recommended by the MIQE guidelines (Bustin S A, Benes V, Garson J A, Hellemans J, Huggett J, Kubista M, et al. The MIQE Guidelines: Minimum Information for Publication of Quantitative Real-Time PCR Experiments. Clin Chem. 26 févr 2009; 55(4):611-22). A minimum of 3 reference genes will be used to insure proper normalization between the conditions. ATF6 activation will be determined through expression of HerpUD, ATF4 through GADD34 and XBP1s using Erdj4.

XBP1 splicing will also be measured by PCR and enzyme digestion, a typical method for this parameter (Fougeray S, Bouvier N, Beaune P, Legendre C, Anglicheau D, Thervet E, et al. Metabolic stress promotes renal tubular inflammation by triggering the unfolded protein response. Cell Death Dis. avr 2011; 2(4):e143). The XBP1 cDNA encompassing the region of restriction site is amplified by PCR. XBP1 PCR products are digested with PstIHF (NEB) restriction enzyme for 1 h at 37° C. The restriction digests are separated on a 1.5% polyacrylamide gel with SYBR® Safe DNA Gel Stain (Invitrogen). The gels are photographed under UV transillumination. The spliced isoform of XBP1 mRNA is resistant to Pst1 and detected as a 448-bp amplification product whereas the unspliced isoform, containing Pst1 restriction site, is detected by the presence of two amplification products of 291-bp and 183-bp.

Device for Preserving an Organ

Another aspect of the present invention relates to a device for preserving an organ, said device comprising an organ container filled with a preservation solution, characterized in that said device further comprises one or more mean for injecting one or more compound into the organ container.

In one embodiment, the injected compound is a therapeutic compound.

In a further embodiment, the injected compound is an activator of PERK-ATF4 pathway, an inhibitor of the ATF6 pathway or an inhibitor of the RNase activity of IRE1a.

In a particular embodiment, the device according to the invention comprises one mean for injecting an activator of PERK-ATF4 pathway into the organ container, one mean for injecting an inhibitor of the ATF6 pathway into the organ container and one mean for injecting an inhibitor of the RNase activity of IRE1α into the organ container.

In one embodiment, the device according to the invention comprises an alarm which gives the health professional notice of the administration moment of the compound by the injected mean.

In one embodiment, the device according to the invention comprises one alarm per compound to be injected.

In one embodiment, the device according to the invention is programmable in order to administer automatically the compound by the injected mean when needed/programmed.

In a particular embodiment, as described in FIG. 7, the device (1) according to the invention comprises an organ container (2), a computing system (3) and three means (4, 5, 6) for injected three compounds.

The organ container (2) is a sterile receptacle for the organ. The organ container is filled with a preservation solution.

The computing system (3), or similar electronic computing device, is adapted to manipulate and/or transform data represented as physical, such as electronic, quantities within the computing system's registers and/or memories into other data similarly represented as physical quantities within the computing system's memories, registers or other such information storage, transmission or display devices. The computing system (3) comprises a display unit for data presentation and data entry.

The mean (4, 5, 6) for injecting a compound comprises a container containing the compound and a device permitting the injection of the compound into the organ chamber. For example, the mean 4 (and 5, 6) is a syringe.

In one embodiment, the device (1) comprises a software. The software permits the implementation of the method according to the invention and plays a role of coordination of the injection times of the compounds to be injected. More particularly, three main steps carried out by the software are: a step of choosing the program or manually entering each compound injection time, a step of starting time count and one or more step of injecting one or more compound.

In a particular embodiment, as described in FIG. 8, the steps carried out by the software are a step of choosing the program or manually entering each compound injection time (S1), a step of starting time count (S2), a step of injecting the first compound (S3) and, if cold ischemia time exceeds 8 hours, a step of injecting the second compound (S4) and a step of injecting the third compound (S5). It results to a final step S6 corresponding to a well-conserved organ ready to be transplanted.

At step S1, the health professional chooses a program (for example, a program adapted to the stored organ) or manually enters the injection time of each compound to be injected. This step has to be carried out immediately after transferring the organ to the organ container.

At step S2, time count automatically starts. The time count may be displayed on the display unit of the device.

At step S3, the injection of the first compound to be injected is automatically carried out at the desired time. Said desired time is planned automatically via the software or manually entered by the health professional at step S1.

If cold ischemia time is inferior to 8 hours, step S6 is directly reached, resulting in a well-conserved organ ready to be transplanted. If cold ischemia time is superior to 8 hours, steps S4 and S5 are carried out.

At step S4, the injection of the second compound to be injected is automatically carried out at the desired time. Said desired time is planned automatically via the software or manually entered by the health professional at step S1.

At step S5, the injection of the third compound to be injected is automatically carried out at the desired time. Said desired time is planned automatically via the software or manually entered by the health professional at step S1.

For each steps S3, S4 and S5, the compound injection is made by the three means 4, 5, 6 respectively.

Performing steps S1, S2 and S3, or S1, S2, S3, S4 and S5, depending on the cold ischemia time, results on step 6 corresponding to a well-conserved organ ready to be transplanted.

In one embodiment, the organ container is hermetically sealed against fluid and pressure.

In one embodiment, the device according to the invention further comprises:
One or several circulatory system,
One or several refrigeration mean,
One or several oxygenator,
One or several pump,
One or several filter,
One or several probe or sensor detecting, for instance, temperature, pressure or any compound concentration,
One or several software.

The invention will be further illustrated by the following figures and examples. However, these examples and figures should not be interpreted in any way as limiting the scope of the present invention.

### FIGURES

FIG. 1. ATF6 is activated during both ischemia and reperfusion. (A) ATF6 protein expression. HAEC were subjected to different durations of ischemia (0 h, 6 h, 12 h, or 24 h). Respective protein expression levels relative to GAPDH protein expression from three independent experiments. Statistics: #: p<0.05 to HO. (B) ATF6 mRNA expression. HAEC were subjected to different durations of ischemia followed or not by 2 h of reperfusion. mRNA levels of ATF6 were quantified by RT-qPCR relatively to untreated HAEC (Control) (N=3). (C) mRNA expression of ATF6 target. HAEC were subjected to different durations of ischemia followed or not by 2 h of reperfusion. mRNA levels of HerpUD were quantified by RT-qPCR relatively to untreated HAEC (Control) (N=3). Statistics: #: p<0.05 to Control. The indicated p value corresponds to the comparison between the indicated group and Control. A.U.=Arbitrary Units, CS=Control Shift, HO=Untreated HAEC, Hx=HAEC subjected to x hours of ischemia, Nx=Independent sample No. x, P=Probe alone. Normally distributed data were analyzed with one-way ANOVA followed by pairwise t-test with pooled SD with Bonferroni correction. Non-normally distributed data were analyzed with Kruskal-Wallis test followed by a Dunn's post-hoc test. For comparisons between two groups, a Wilcoxon-Mann-Whitney test was performed.

FIG. 2. ATF4 is detected during the early phase of ischemia. (A) ATF4 protein expression. HAEC were subjected to different durations of ischemia (0 h, 6 h, 12 h, or 24 h). Respective protein expression levels relative to GAPDH protein expression from three independent experiments. Statistics: #: p<0.05 to HO. (B) ATF4 mRNA expression. HAEC were subjected to different durations of ischemia followed or not by 2 h of reperfusion. mRNA levels of ATF4 were quantified by RT-qPCR relatively to untreated HAEC (Control) (N=3). (C) mRNA expression of ATF4 target. HAEC were subjected to different durations of ischemia followed or not by 2 h of reperfusion. mRNA levels of GADD34 were quantified by RT-qPCR relatively to untreated HAEC (Control) (N=3). Statistics: #: p<0.05 to Control. The indicated p value corresponds to the comparison between the indicated group and Control. A.U.=Arbitrary Units, CS=Control Shift, HO=Untreated HAEC, Hx=HAEC subjected to x hours of ischemia, Nx=Independent sample No. x, P=Probe alone. Normally distributed data were analyzed with one-way ANOVA followed by pairwise t-test with pooled SD with Bonferroni correction. Non-normally distributed data were analyzed with Kruskal-Wallis test followed by a Dunn's post-hoc test. For comparisons between two groups, a Wilcoxon-Mann-Whitney test was performed.

FIG. 3. ATF6 has a pro-death role for endothelial cells during ischemia reperfusion. (A) Validation of AEBSF. HAEC were exposed during 6 h to tunicamycin (TM) alone or in combination with AEBSF (TM+AEBSF), an inhibitor of S1P involved in the activation cleavages of ATF6. mRNA levels of HerpUD and GRP78 were quantified by RT-qPCR relatively to untreated HAEC (Control) (N=3). Statistics: #: p<0.05 to Control; *: p<0.05 between indicated groups. (B) AEBSF improves viability. XTT viability assay on HAEC subjected to 24 h of ischemia followed by 6 h of reperfusion relatively to untreated HAEC (CTL). During hypoxia, HAEC were untreated (CTL), subjected to 24 h of ischemia followed by 6 h of reperfusion with (AEBSF) or without (HR) the addition of AEBSF, an inhibitor of S1P involved in the activation cleavages of ATF6 (N=3, n=3). Statistics: #: p<0.05 to CTL; *: p<0.05 between indicated groups. (C) AEBSF decreases CHOP expression. During 6 h, HAEC were exposed to tunicamycin (TM), AEBSF (AEBSF), or both (TM+AEBSF). mRNA levels of CHOP were quantified by RT-qPCR relatively to untreated HAEC (Control) (N=3). (D) Validation of the silencing of ATF6 on HAEC subjected to 24 h of ischemia by RT-qPCR from three independent experiments. Statistics: #: p<0.05 to siCTL. (E) siATF6 improves viability. Viability of HAEC transfected with scrambled siRNA (siCTL) or siRNA targeting ATF6 (siATF6) and subjected to ischemia-reperfusion. Control HAEC were transfected with siCTL but not subjected to 24 h of ischemia followed by 6 h of reperfusion (N=5, n=3). Statistics: #: p<0.05 to siCTL. (F) CHOP expression is dependent on ATF6. HAEC transfected with scrambled siRNA (siCTL H24) or siRNA targeting ATF6 (siATF6 H24) were subjected to 24 h of ischemia or not (siCTL HO). mRNA levels of CHOP were quantified by RT-qPCR relatively to siCTL-transfected HAEC unexposed to 24 h of ischemia (siCTL HO) (N=3). Statistics: #: p<0.05 to CTL; *: p<0.05 between indicated groups. Statistics: #: p<0.05 to CTL; *: p<0.05 between indicated groups. Normally distributed data were analyzed with one-way ANOVA followed by pairwise t-test with pooled SD with Bonferroni correction. Non-normally distributed data were analyzed with Kruskal-Wallis test followed by a Dunn's post-hoc test. For comparisons between two groups, a Wilcoxon-Mann-Whitney test was performed.

FIG. 4. The eIF2α-ATF4 axis is pro-survival during ischemia reperfusion. (A) Salubrinal improves viability. XTT viability assay on HAEC subjected to 24 h of ischemia followed by 6 h of reperfusion relatively to untreated HAEC (CTL). During hypoxia, HAEC were either not pharmacologically-treated (HR) or treated with Salubrinal (N=3, n=3). Statistics: #: p<0.05 to CTL; *: p<0.05 between indicated groups. (B) Validation of the silencing of PERK and ATF4 on HAEC subjected to 24 h of ischemia by RT-qPCR from three independent experiments. Statistics: #: p<0.05 to siCTL. (C) siPERK and siATF4 do not alter viability. Viability of HAEC transfected with scrambled siRNA (siCTL) or siRNA targeting either PERK (siPERK) or ATF4 (siATF4) and subjected to ischemia-reperfusion. Control HAEC were transfected with siCTL but not subjected to 24 h of ischemia followed by 6 h of reperfusion (N=3, n=3). The indicated p values correspond to the comparison between the indicated group and siCTL. Normally distributed data were analyzed with one-way ANOVA followed by pairwise t-test with pooled SD with Bonferroni correction. Non-normally distributed data were analyzed with Kruskal-Wallis test followed by a Dunn's post-hoc test. For comparisons between two groups, a Wilcoxon-Mann-Whitney test was performed.

FIG. 5. Silencing either IRE1α or XBP1 decreases endothelial cell survival while IRE1α's RNase inhibition protects them from ischemia reperfusion injury. (A) STF083010 improves viability. XTT viability assay on HAEC subjected to 24 h of ischemia followed by 6 h of reperfusion relatively to untreated HAEC (CTL). During hypoxia, HAEC were either not pharmacologically-treated (HR) or treated with STF083010 (STF), an inhibitor of IRE1α's RNase activity (N=3, n=3). Statistics: #: p<0.05 to HR; *: p<0.05 between indicated groups. (B) siIRE1α and siXBP1 decrease viability. Viability of HAEC transfected with scrambled siRNA (siCTL) or siRNA targeting either IRE1α (siIRE1α) or XBP1 (siXBP1) and subjected to ischemia-reperfusion. Control HAEC were transfected with siCTL but not subjected to 24 h of ischemia followed by 6 h of reperfusion (N=3, n=3). Statistics: #: p<0.05 to siCTL. (C) Decreased survival of IRE1α knocked-out cells during HR. Murine embryonic cells (MEC) knocked-out (−/−) or knocked-out but rescued by a retrovirus containing the Flag-tagged human IRE1α (+/+) were subjected to 24 h of ischemia followed by 6 h of reperfusion before cell survival assessment by XTT assay (N=3, n=3). Survival rate was normalized to untreated MEF (or MEC) of their corresponding genotype. Statistics: #: p<0.05 to IRE1α^{+/+} subjected to HR. (D) STF083010 decreased CHOP mRNA expression. HAEC were subjected to 24 h of ischemia with or without the addition of STF083010. mRNA levels of CHOP were quantified by RT-qPCR relatively to siCTL-transfected HAEC unexposed to 24 h of ischemia (Hypoxia −) (N=3). (E) siXBP1 does not alter CHOP mRNA expression. HAEC transfected with scrambled siRNA (siCTL H24) or siRNA targeting XBP1 (siXBP1 H24) were subjected to 24 h of ischemia or not (siCTL HO). mRNA levels of CHOP were quantified by RT-qPCR relatively to siCTL-transfected HAEC not exposed to 24 h of ischemia (siCTL HO) (N=3). Statistics: #: p<0.05 to siCTL HO. Statistics: #: p<0.05 to Hypoxia −; *: p<0.05 between indicated groups. Normally distributed data were analyzed with one-way ANOVA followed by pairwise t-test with pooled SD with Bonferroni correction. Non-normally distributed data were analyzed with Kruskal-Wallis test followed by a Dunn's post-hoc test. For comparisons between two groups, a Wilcoxon-Mann-Whitney test was performed.

FIG. 6: Schematic representation of UPR involvement with CIT. A: ATF4 is only activated during the early phase of ischemia, at times corresponding to a minimal level of clinical complications, and if activation is maintained then survival is increased. B: ATF6 is activated later, at times generally associated with increased delayed graft function and long term complications; if inhibited, survival is increased. C: IRE1α RNase activation is not detected before reperfusion, however it is only observable when preceded by at least 12 hours of ischemia, hence also related to the level of injury; if it is inhibited, survival will increase. D: ATF6 and IRE1α RNase appear to link to cell death through CHOP, in a CIT-dependent manner.

FIG. 7: Schematic representation of the device for preserving an organ.

FIG. 8: Software process steps.

### EXAMPLE

Material & Methods

Transmission Electronic Microscopy.

Cells or tissues were fixed with 3% glutaraldehyde during 2 h at 4° C. Cells were scraped before performing a 15-min centrifugation at 500 g. Cells were then resuspended in PBS (1:10 volume). A post-fixation step was achieved with OsO₄ 1% during 1 h at 4° C. The dehydration was obtained by processing the cells in increasing acetone concentrations. Then, cells were fixed in araldite resin before a polymerization step of 24 h at 60° C. Semithin sections (1 μm) were stained with toluidine blue to distinguish cells. Then ultrathin sections (˜600 nm) were obtained with ultramicrotome Ultracut S (Reichert). To enhance contrasts, uranyle acetate and lead salts were used. The cells were then observed by Transmission Electronic Microscopy (TEM) using JEOL 1010 microscope.

Cell Culture.

Primary human aortic endothelial cells (HAEC) were obtained from Gibco (Lot No: #765093 and Lot No: #999999) and cultured on 1% gelatin (Sigma) coated flasks of 75 cm² in M200 medium supplemented with LSGS (Gibco), 8% fetal bovine serum (FBS), and 100 μg/mL penicillin and streptomycin in a humidified atmosphere at 21% O₂, 5% CO2 and 37° C. The cells were split at a ratio of 1:4 every 5 days. Cells up to passage 5 were used in this study. All cell culture media, serum, and supplements were purchased from Invitrogen. STF083010 (10 μM) and 4μ8c (10 μM) were purchased from Axon MedChem. Salubrinal (75 μM) was from R&D. Tunicamycin (2 μg/L), sodium 4-phenylbutyrate (1 mM), and 4-(2-Aminoethyl)benzenesulfonyl fluoride (AEBSF, 300 μM) were from Sigma. All chemicals were dissolved in DMSO (Sigma). IRE1α^{-/-} and IRE1α^{+/+}—which are IRE1α^{-/-} cells rescued by a retrovirus encoding a Flag-tagged human IRE1α—murine embryonic cells (MEC) were produced as previously described (Volmer R, Ploeg K van der, Ron D. Membrane lipid saturation activates endoplasmic reticulum unfolded protein response transducers through their transmembrane domains. Proc. Natl. Acad. Sci. 2013; 201217611). MEC were cultured in DMEM high-glucose media (Gibco) supplemented with 10% FBS, non-essential amino acids (Gibco), penicillin and streptomycin (Gibco), L-glutamine (Gibco), and puromycin (3 μg/mL).

Animal Model

Surgical and experimental protocols were performed in accordance with French Ministry of Agriculture, National Institute for Agronomic Research and Poitou Charentes ethical comity of animal experimentation (protocol number CE2012-4). We used 3 months old Large White pigs (40±4 kg, IBiSA, INRA Magneraud, France). As previously described (Hauet T, Goujon J M, Vandewalle A, Baumert H, Lacoste L, Tillement J P, et al. Trimetazidine reduces renal dysfunction by limiting the cold ischemia/reperfusion injury in autotransplanted pig kidneys. J. Am. Soc. Nephrol. JASN. 2000; 11:138-48), after anesthesia the left kidney was approached through a midline abdominal incision. The left renal vascular pedicle and the ureter were atraumatically dissected, and the kidney was then removed and flushed with 4° C. University of Wisconsin preservation solution (UW) supplemented with 5000 IU/L UFH and stored at 4° C. for 24 h. Cortical samples were collected at the indicated intervals.

Hypoxia-Reoxygenation Experiments.

HAEC were washed with PBS before being incubated in cold University of Wisconsin (UW) preservation solution in a chamber with hypothermic (4° C.) hypoxic atmosphere obtained by flushing the chamber atmosphere with Bactal 2 (0% O₂, 95% N₂ and 5% CO₂) until the reach of 0% 02 in the chamber atmosphere. The oxygen level was controlled with the oximeter Oxy-4 micro from PreSens Precision Sensing GmbH with channels present in the outside atmosphere, the chamber atmosphere, and the cell supernatant. To perform the normothermic reoxygenation, cells were washed with PBS and then incubated with M200 supplemented with 2% FBS in a humidified atmosphere at 21% O₂, 5% CO₂ and 37° C. The durations of both hypoxia and reoxygenation are indicated in figure legends and in the manuscript.

Quantitative PCR (RT-qPCR).

RNA was extracted from HAEC using the NucleoSpin RNA kit (Macherey-Nagel) containing a DNase treatment to remove potentially contaminating genomic DNA. RNA quality was verified by resolution on a 1.5% (wt/vol) agarose gel and measurement of A₂₆₀ₙₘ/A₂₈₀ₙₘ and A₂₆₀ₙₘ/A₂₃₀ₙₘ ratios using a NanoDrop™ 2000 (Thermo Scientific). A quantity of 1 μg of total RNA was reverse-transcribed with High Capacity cDNA Reverse Transcription kit (ABsystems). RT-qPCR was performed in triplicate with negative template controls, negative enzyme controls and the use of a calibrator to limit inter-run variations as recommended by the MIQE guidelines (Bustin S A, Benes V, Garson J A, Hellemans J, Huggett J, Kubista M, et al. The MIQE Guidelines: Minimum Information for Publication of Quantitative Real-Time PCR Experiments. Clin. Chem. 2009; 55:611-22). We validated two reference genes, RPS5 and RPS15, to normalize the mRNA levels of each target gene under hypoxia and hypoxia-reoxygenation conditions by using the geNorm algorithm (Vandesompele J, Preter K D, Pattyn F, Poppe B, Roy N V, Paepe A D, et al. Accurate normalization of real-time quantitative RT-PCR data by geometric averaging of multiple internal control genes. Genome Biol. 2002; 3:research0034). The runs were performed with Rotor-Gene 3000 (Qiagen) and results were analyzed using EasyqpcR package (Le Pape, S. EasyqpcR: EasyqpcR for easy analysis of real-time PCR data at IRTOMIT-INSERM U1082 R package, IRTOMIT-INSERM U1082. sylvain.le.pape@univ-poitiers.fr.2012).

PCR and XBP1 mRNA Splicing by Enzyme Digestion.

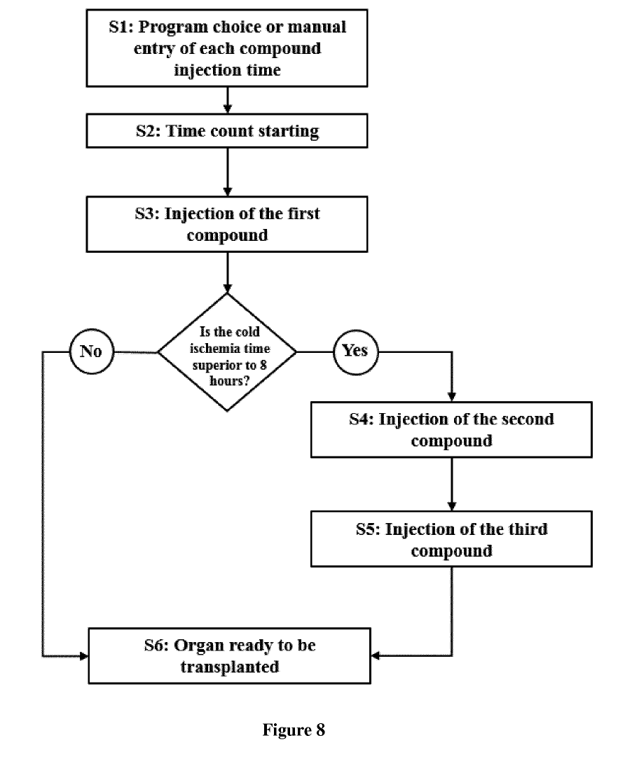

We followed the method used in (Fougeray S, Bouvier N, Beaune P, Legendre C, Anglicheau D, Thervet E, et al. Metabolic stress promotes renal tubular inflammation by triggering the unfolded protein response. Cell Death Dis. 2011; 2:e143). The XBP1 cDNA encompassing the region of restriction site was amplified by PCR. XBP1 PCR products were digested with PstI^{HF} (NEB) restriction enzyme for 1 h at 37° C. The restriction digests were separated on a 1.5% polyacrylamide gel with SYBR® Safe DNA Gel Stain (Invitrogen). The gels were photographed under UV transillumination. The spliced isoform of XBP1 mRNA was resistant to Pst1 and was detected as a 448-bp amplification product whereas the unspliced isoform, containing Pst1 restriction site, was detected by the presence of two amplification products of 291-bp and 183-bp (FIG. 8A, right).

Immunoblotting.

HAEC were washed in cold PBS and resuspended in complete lysis buffer (Roche Diagnostics, #04719956001) before being sonicated at output power of 2 for 3 seconds (Branson Sonifier 450). The protein concentration of cell lysate supernatants was measured by BCA protein assay (Bio-Rad, #23225). 10-20 μg of cell lysate were applied to SDS/PAGE with Criterion™ Tris-HCl Precast Gels (Bio-Rad) and transferred to Hybond PVDF membrane (Amersham Biosciences), followed by standard Western-blot procedure. The bound primary antibodies were detected with ChemiDoc™ MP imager (Bio-Rad) by the use of HRP-conjugated secondary antibody and the ECL detection system (Amersham Biosciences). The band density was semiquantified with Image Lab Software (Bio-Rad). We used primary antibodies from Cell Signaling for PERK (#3192, 1:1000) and IRE1α (#3294, 1:1000); from Abcam for both XBP1 isoforms (ab37152, 1:500), ATF4 (ab1371, 1:1000), and ATF6 (ab37149, 1:1000) and from Millipore for GAPDH (CB1001, 1:30000). The secondary antibodies were from Invitrogen (G-21040 and G-21234, 1:4000) and Santa-Cruz (sc-2922, 1:4000).

siRNA Transfection and Cell Viability Assay.

Reverse transfection were performed using ON-TARGETplus SMART pool siRNAs targeting IRE1α (#L-004951-02-0005), XBP1 (#L-009552-00-0005), PERK (#L-004883-00-0005), ATF4 (#L-005125-00-0005), ATF6 (#L-009917-00-0005), or ON-TARGETplus Non-targeting Pool control siRNAs (#D-001810-10-05). They were purchased from Dharmacon and used at a final concentration of 20 nM. The transfection was performed using Lipofectamine RNAiMAX and Opti-MEM reduced serum medium according to the manufacturer's instructions (Invitrogen). After 6 hours of transfection, the cell medium was replaced with fresh cell culture medium containing 10% of FBS, LSGS but no antibiotics and no antimycotics. After 24 hours of transfection, cells were subjected to hypoxia-reoxygenation experiments. For cells in 96- and 24-wells plates, cell viability was analyzed according to protocol provided (XTT cell proliferation kit II, Roche Diagnostics) while cells in 6-wells were subjected to RT-qPCR analysis to detect knockdown efficiency.

EMSA.

Frozen samples from cultured cells were processed for nuclear and cytoplasmic protein separation by the NE-PER Nuclear and Cytoplasmic Extraction kit (Pierce, Fisher Bioblock #78833) following the manufacturer's recommendations. Nuclear extracts were then processed with the LightShift Chemiluminescent EMSA Kit (Pierce #20148) using biotinylated DNA probes, migration was performed on polyacrylamid TBE gels (Biorad #3450049) and transfer on Zeta-Probe membrane (Biorad #162-0197). Quantification of shift was semiquantified by ImageJ software.

Immunohistofluorescence.

Snap Frozen samples from kidney cortex were cut on a cryotome. Sections were stained with a primary antibodies from Abcam directed against UPR pathways markers: XBP1 (ab37152, 1:500), ATF4 (ab1371, 1:1000), and ATF6 (ab37149, 1:1000). An appropriate secondary antibody combined to Alexa488 fluorescent dye was used to detect the signal.

Microarray Data Collection and Analysis.

We acquired raw microarray data and clinical informations for kidney transplant patients which grafts had been biopsied at time of procurement (HO), at the end of preservation and 60 min after reperfusion (R60) from the Gene Expression Omnibus (GEO (Edgar R, Domrachev M, Lash A E. Gene Expression Omnibus: NCBI gene expression and hybridization array data repository. Nucleic Acids Res. 2002; 30:207-10)(Barrett T, Wilhite S E, Ledoux P, Evangelista C, Kim I F, Tomashevsky M, et al. NCBI GEO: archive for functional genomics data sets—update. Nucleic Acids Res. 2013; 41:D991-5), direct link to database: http://www.ncbi.nlm.nih.gov.gate2.inist.fr/geo/query/acc.cgi?acc=%20GSE43974). Data was analyzed using the GEO2R tool (Davis S, Meltzer P S. GEOquery: a bridge between the Gene Expression Omnibus (GEO) and BioConductor. Bioinforma. Oxf. Engl. 2007; 23:1846-7).

Statistics.

Data were analyzed using NCSS software (Hintze, Jerry. NCSS 2007. NCSS, LLC. Kaysville, Utah, USA. www.ncss.com. 2007) and are expressed as the mean±SD. Data normality was assessed by performing a Shapiro-Wilk W Test. Normally distributed data were analyzed with one-way ANOVA followed by pairwise t-test with pooled SD with Bonferroni correction. Non-normally distributed data were analyzed with Kruskal-Wallis test followed by a Dunn's post-hoc test. For comparisons between two groups, a Wilcoxon-Mann-Whitney test was performed. A p value<0.05 was considered statistically significant.

Results

The UPR is Part of the Relationship Between CIT and Level of Injury

We used a preclinical model of kidney transplantation on 35-40 kg Large White pigs, presenting a high level of similarity to humans in terms of the size, anatomy and physiology. We subjected the kidneys to different lengths of cold ischemia, and observed the cell alterations using electron microscopy. In accordance with previous studies, we showed that (data not shown) mitochondria are severely impacted by cold ischemia duration (swelling and loss of cristae). However, the endoplasmic reticulum (ER) was also altered, with a clear detachment of the ribosomes from the ER membrane—a known sign of ischemic lesions.

This led us to investigate the ER stress pathways: the UPR. These pathways are necessary for the cell's adaptation to stresses and are acknowledged as critical mediators of diseases. Accumulation of unfolded proteins in the ER lumen triggers the UPR, composed of the IRE1α-XBP1, PERK-eIF2α-ATF4, and ATF6 pathways. Each respectively activates the transcription factors XBP1s, ATF4, and ATF6; regulating a broad spectrum of UPR-related genes involved in phospholipid synthesis, ER-associated degradation (ERAD), protein quality control and amino acid and redox metabolisms.

We endeavored to characterize the dynamics of UPR activation during cold ischemia. As the emblematic protein of each pathway (ATF4, ATF6, and XBP1s) are transcription factors, we performed electro-mobility shift assay (EMSA) to determine if they translocated to the nucleus during cold storage (data not shown). Our results show that each pathway has a different relationship to cold ischemia time: -ATF4 is translocated early (6-12 hours), then is absent from the nucleus in case of prolonged CIT; -ATF6 is present at basal levels until 6 hours, then the translocation signal increased during the last 12 hours; -XBP1s could not be detected at any time point. We then performed immunofluorescent detection.

ATF4 was detected in the preserved organs during the first 12 hours (data not shown) then staining was weaker. ATF6 was present at the beginning of storage and increases greatly in response to CIT, with the highest levels detected at the end of 24 h (data not shown). XBP1 staining was absent at the beginning then a weak signal was detected at 12 and 24 hours (data not shown), however intense noise rendered the analysis difficult. Finally, we stained for CHOP, a pro-apoptotic factor induced by the UPR, and showed it was absent in early samples then its expression increased with CIT (data not shown).

Our preclinical findings suggest a deep involvement of the UPR in the relationship between CIT and organ quality, each pathway demonstrating a specific kinetic. To investigate this phenomenon further, we established an in vitro model of cold ischemia.

Endothelial Cells Subjected to Cold Ischemia and Warm Reperfusion Present Common Features with Preserved Organs.

We subjected primary endothelial cells (EC) to increasing durations of cold storage with the same preservation solution used in the clinic and in our preclinical model. We measured their viability after 6 hours of reperfusion mimicked by restoring normal culture conditions (temperature, atmosphere, medium). There was a distinct association between CIT and survival, trending towards lower survival after 6 and 12 hours of preservation, reaching a significant level of death (49%±2% up to 65%±2%) after 24 h of cold storage (data not shown). Electron microscopy on 24 h preserved cells (H24) showed swelling and loss of cristae of the mitochondria and alterations of the ER with clear ribosome detachment (data not shown).

Thus, our model mimicked the features of organ preservation injury.

The UPR is Activated in Our In Vitro Model of Organ Preservation

ATF6:

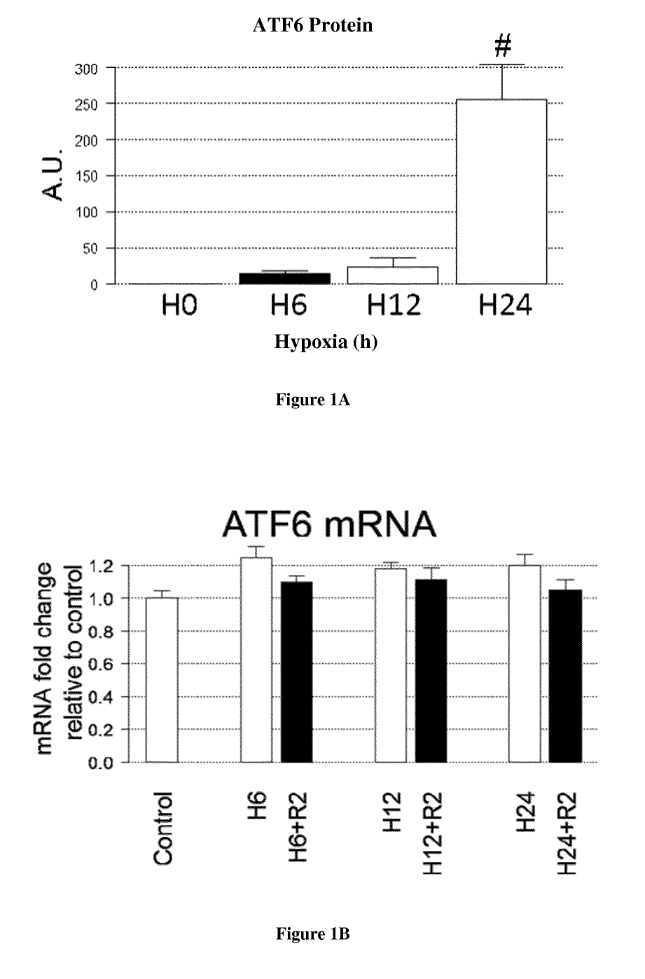

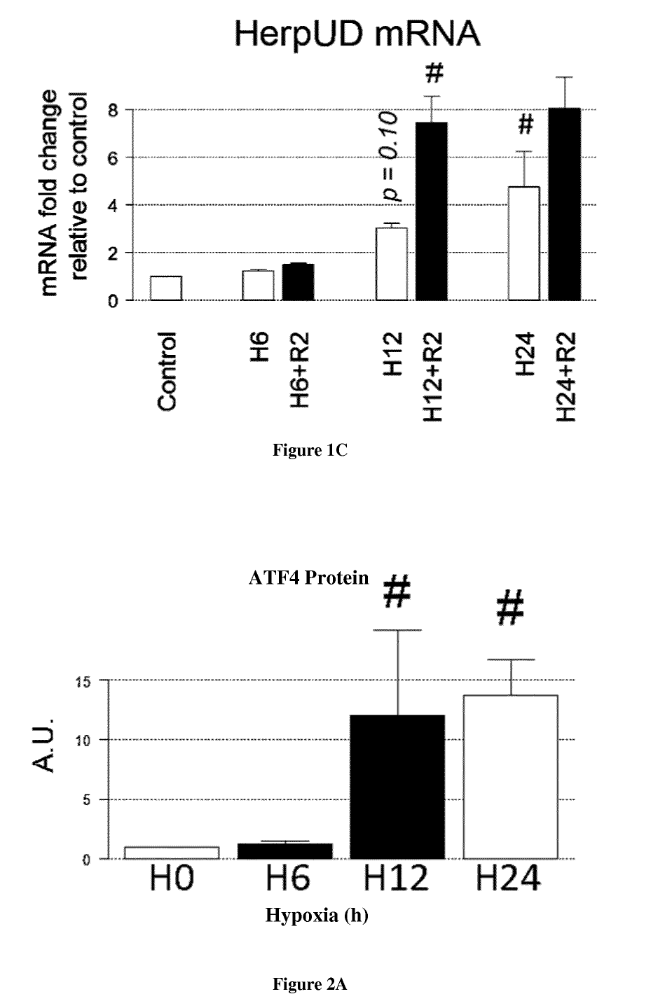

ATF6 protein was detected at 24 h of ischemia (FIG. 1A). However, mRNA levels remained unchanged during both ischemia and reperfusion suggesting a translational or post-translational regulation (FIG. 1B). We detected increased ATF6 nuclear translocation by EMSA from 6 h to 24 h of ischemia with a spike at 18 h (data not shown), matching in vivo observations. To determine functional significance, we analyzed the mRNA levels of an ATF6 downstream target, HerpUD (Shoulders M D, Ryno L M, Genereux J C, Moresco J J, Tu P G, Wu C, et al. Stress-Independent Activation of XBP1s and/or ATF6 Reveals Three Functionally Diverse ER Proteostasis Environments. Cell Rep. 2013; 3:1279-92.) (FIG. 1C). In accordance with ATF6 expression and translocation, we observed increased HerpUD expression from 12 h to 24 h of ischemia, further increased at reperfusion.

ATF4:

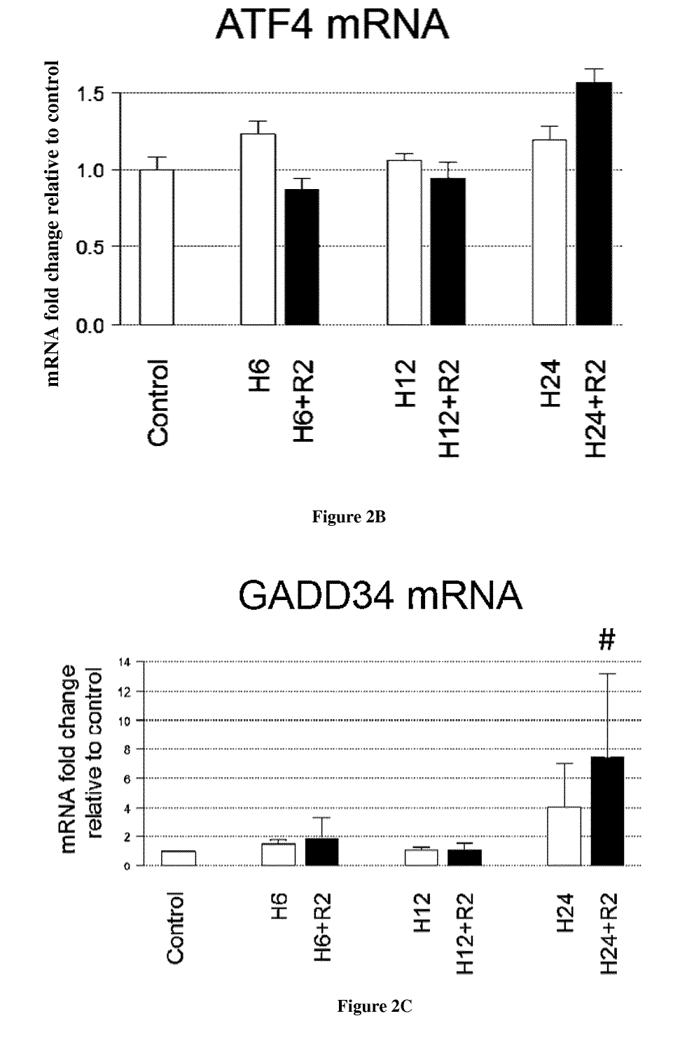

ATF4 protein increased from 12 h to 24 h of ischemia (FIG. 2A). Here also, the unchanged expression level of ATF4 mRNA suggested translational regulation (FIG. 2B). ATF4 nuclear translocation was shown at 0 h and 6 h of ischemia as well as at 18 h of ischemia (data not shown). The mRNA level an ATF4 downstream target, GADD34, tended to increase after 24 h cold ischemia. However, a significant increase could be detected in cells subjected to 24 h cold storage followed by 2 hours of reperfusion (FIG. 2C). These results are at odds with in vivo data, a dichotomy which was investigated further in this study.

XBP1:

cold ischemia alone did not activate XBP1 mRNA splicing (data not shown), however splicing was detected immediately after reperfusion. This is coherent with the weak XBP1s signals recorded in vivo. However, investigating the influence of CIT on XBP1 mRNA splicing at reperfusion showed that it was only detectable if ischemia lasted more than 8 h (data not shown). Activation of IRE1α could thus be a consequence of prolonged CIT. In contrast to the absence of splicing, we observed XBP1s protein at 6 h of ischemia (data not shown) while XBP1u protein expression remained unchanged. Concordant with in vivo data, there was no nuclear translocation of XBP1s during ischemia (data not shown). We analyzed mRNA levels of an XBP1 target, Erdj4 (data not shown): in accordance with splicing data, Erdj4 expression was not modulated during ischemia but it gradually increased at reperfusion in response to CIT length.

As our in vivo data showed increased CHOP staining in a CIT-dependent manner, we measured mRNA expression in our cells (data not shown): CHOP tends to increase towards the end of 24 h cold ischemia, and a significant increase was observed in cells subjected to 2 hours reperfusion after both 12 and 24 h of cold ischemia.

We demonstrated that the UPR was activated during in vitro cold ischemia-reperfusion similarly as was observed in preserved pig kidneys. Each pathway had its own kinetic: ATF6 appeared to be activated by long CIT (>12 h); while IRE1α-XBP1 is only activated at reperfusion, although only if cells have previously been subjected to at least 8 h of CIT; the PERK-ATF4 branch is more difficult to define, as both early and late activations are recorded. CHOP expression also appears induced by elongated CIT, as per in vivo data. We then endeavored to determine if these pathways played a role in cell survival.

ATF6 has a Pro-Death Role.

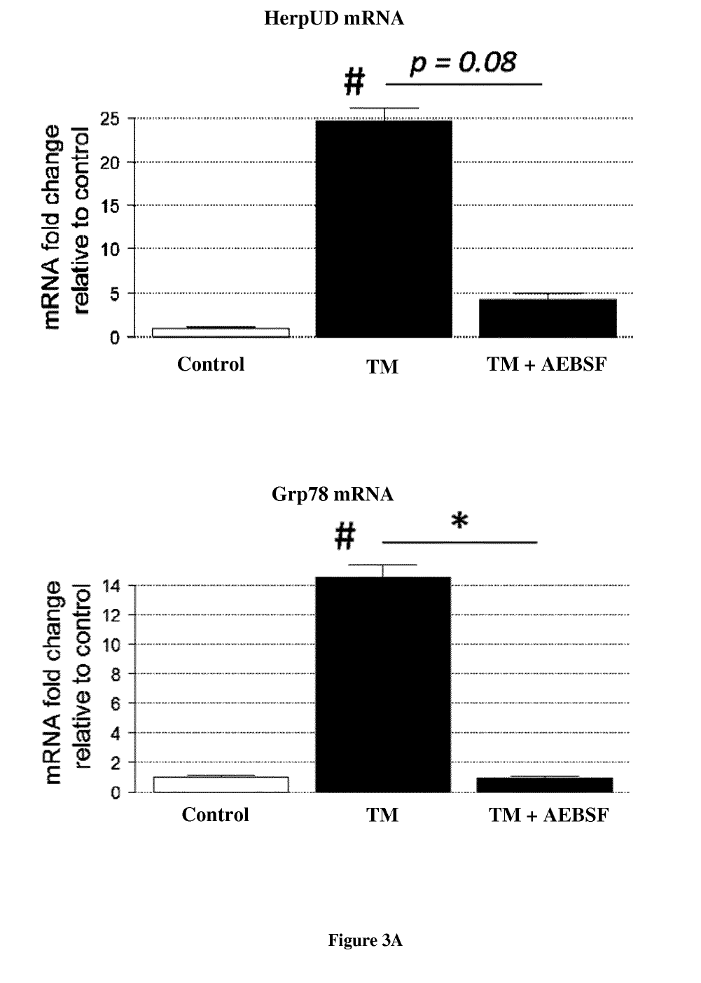

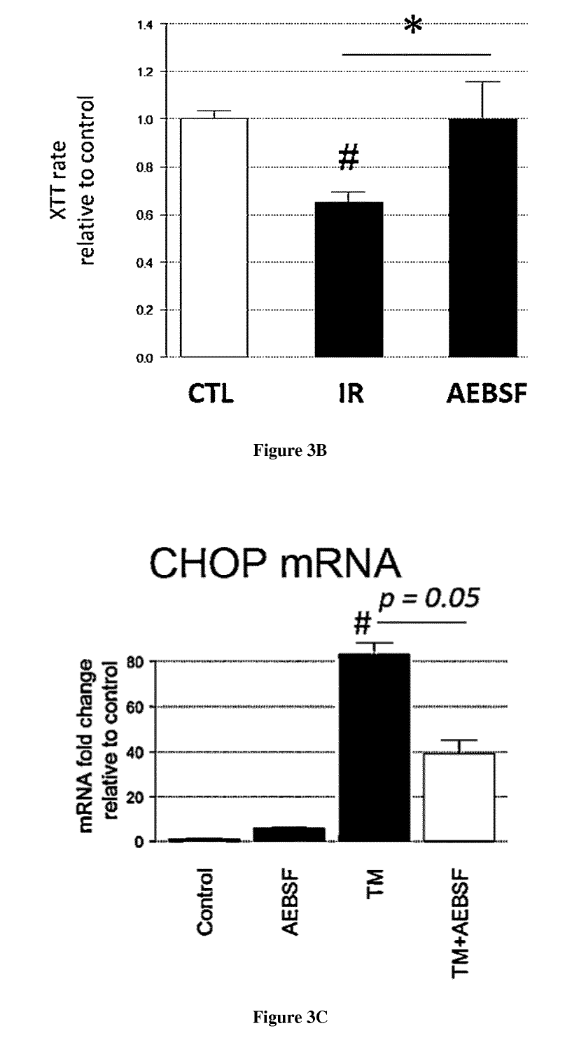

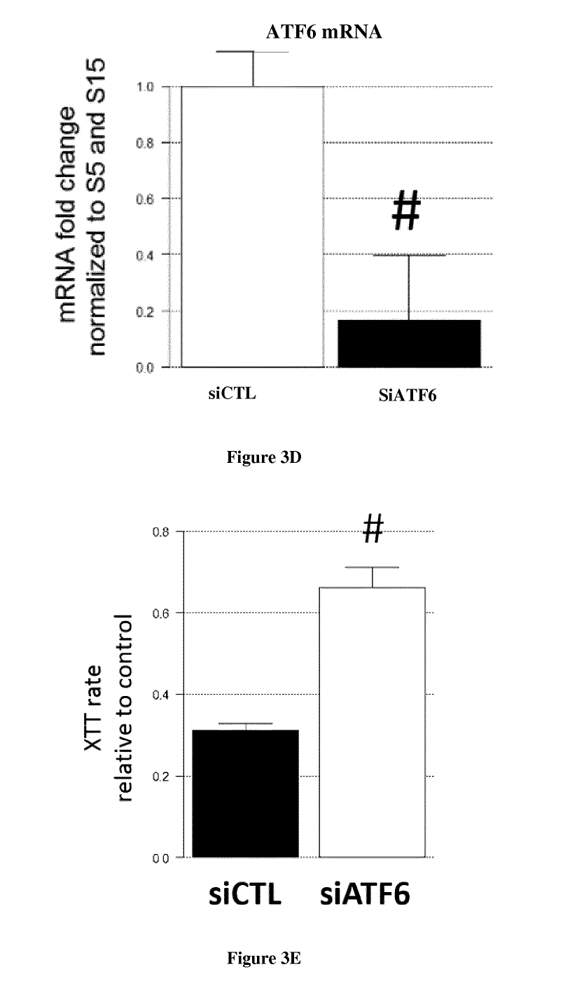

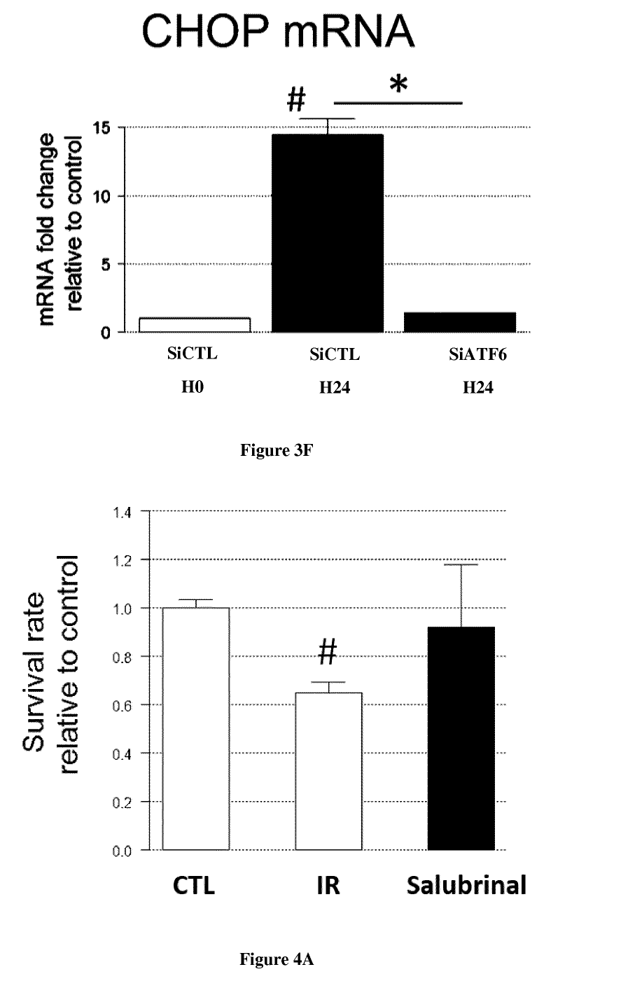

To understand the role of ATF6 activation, we modulated ATF6 signaling. AEBSF is a chemical inhibitor of S1P and S2P proteases and correctly decreased mRNA expression of ATF6 downstream targets HerpUD and GRP78 after a 6 h-tunicamycin treatment (FIG. 3A). This inhibition protected endothelial cells against cold ischemia-induced death (FIG. 3B). As we showed that prolonged CIT associated with increased expression of pro-apoptotic CHOP, which can be regulated by ATF6, we validated that inhibition of ATF6 by AEBSF could indeed lower CHOP mRNA expression when the UPR was induced by tunicamycin (FIG. 3C).

We completed the ATF6 pathway study using a siRNA targeting ATF6, as AEBSF is not sufficiently specific. Our siRNA correctly decreased the expression of both ATF6 mRNA and protein (FIG. 3D). SiRNA-mediated knockdown of ATF6 (FIG. 3E) improved cell survival. Regarding the regulation of the pro-apoptotic factor CHOP, the use of siATF6 significantly decreased its expression (FIG. 3F). Altogether, these results suggest that the late activation of the ATF6 pathway is linked with increased cell death, likely through CHOP regulation.

To investigate ATF6 kinetics in relation to CIT, we modulated the pathway with AEBSF at different times after the start of cold ischemia (data not shown). While AEBSF treatment at HO correctly protected EC against death, there was a trend towards decreased protection when treating only at 6 h. The protective effect was significantly lost when we waited 12 h before treatment. This confirms the observation that ATF6 activation is associated with prolonged CIT and linked to the increased cell death observed when CIT exceeds 8 hours.

Activation of the ATF4 Pathway Protects Endothelial Cells from IRI.

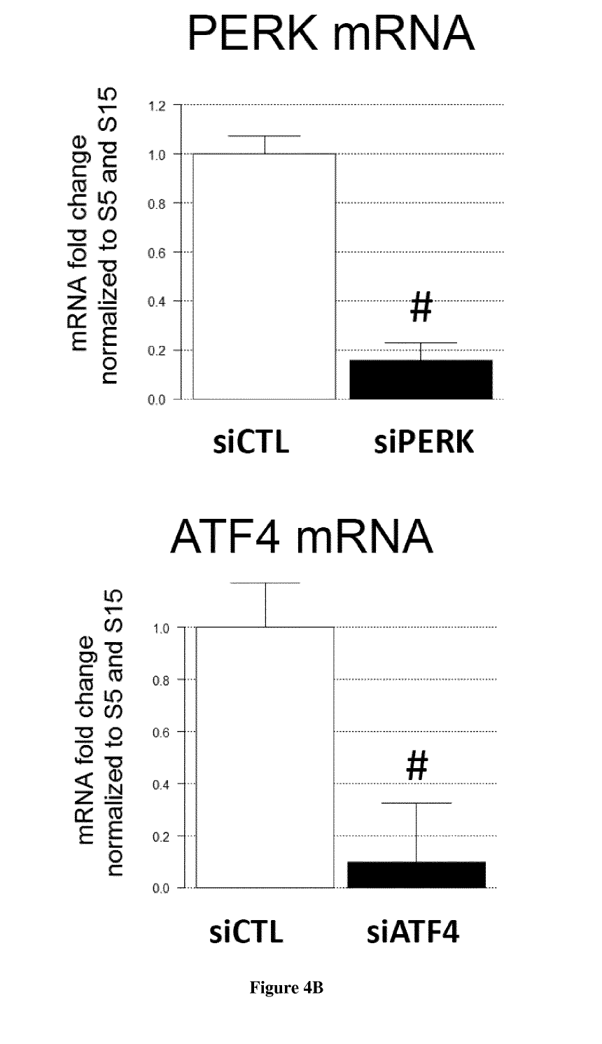

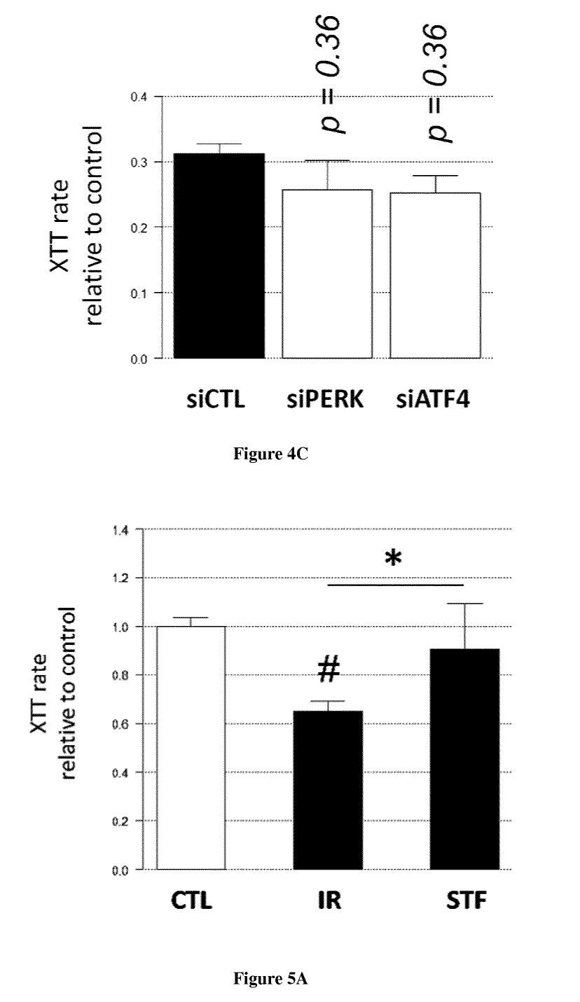

We modulated ATF4 signaling during hypoxia with Salubrinal, an inhibitor of eIF2a's dephosphorylation, maintaining the PERK-eIF2α-ATF4 pathway activated. Salubrinal effectively increased ATF4 production in EC subjected to 6 h of ischemia (data not shown) and significantly increased EC survival after IR (FIG. 4A). Hence, activation of PERK-ATF4 appears to be protective against cell death.

To further investigate this, we used siRNA targeting either PERK or ATF4. These proteins were effectively silenced by their respective siRNA both at the mRNA and protein levels (FIG. 4B). Use of these siRNA during ischemia did not alter cell fate (FIG. 4C), suggesting that the eIF2α-ATF4 axis offers protection against IR only if its activation is maintained.

As we observed discrepancy between in vivo and in vitro data, notably regarding PERFK-ATF4 activation kinetic in relation to CIT, we treated cells with Salubinal at different times after the start of cold ischemia (data not shown). While treatment during all 24 h of preservation significantly protected cells against death, this was lost if the treatment was delayed for 6 hours. Thus, it seems PERK-ATF4 induction during the early hours of preservation is important for cell survival and can be protective if its activation in maintained. This is concordant with the early activation observed in vivo. Hence, the late activation observed in vitro appears to be unrelated to cell survival.

The IRE1α-XBP1 Pathway has a Dual Effect on Cell Survival.

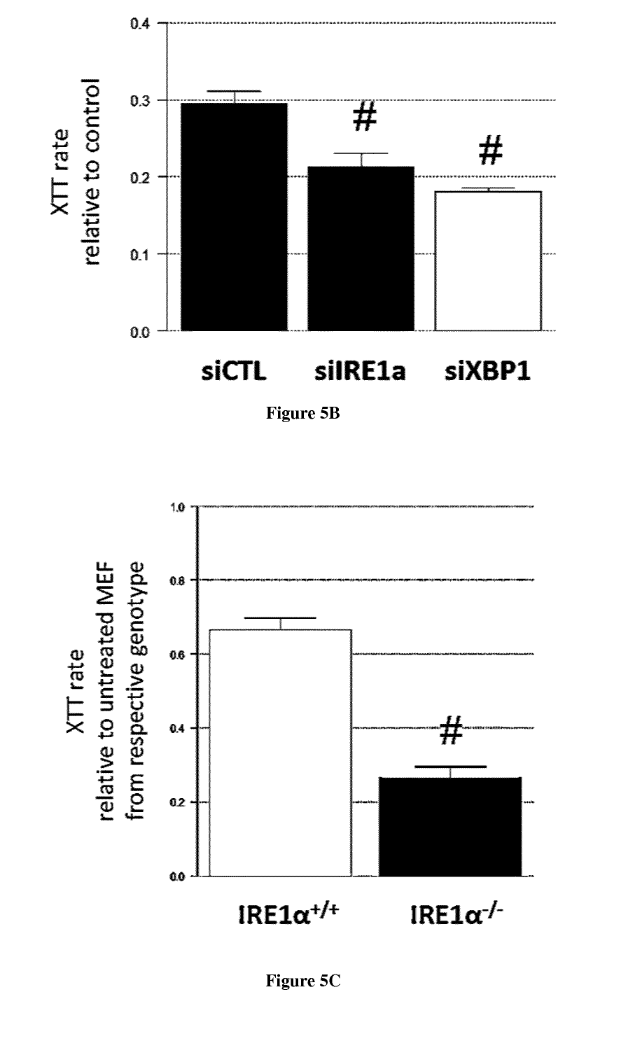

We modulated the IRE1α-XBP1 pathway with STF083010 (STF), a chemical inhibitor of IRE1α's RNase activity. STF abrogated XBP1s and Erdj4 expression in tunicamycin-stimulated cells (data not shown) and inhibited splicing in cells subjected to 6 h ischemia (data not shown). STF treatment significantly increased EC survival after IR (FIG. 5A), indicating a pro-death role for IRE1α's RNase activity. Here also, the activation kinetic of this pathway was investigated by delaying treatment with STF (data not shown): delaying treatment by 6 and 12 hours did not alter the protection offered by STF, confirming a late activation of this pathway.

To investigate this phenomenon further we used siRNAs against both IRE1a and XBP1. The efficacy of each siRNA was verified at both mRNA and protein levels (data not shown). Surprisingly, knockdown of either IRE1α or XBP1 significantly decreased cell survival in our in vitro model instead of protecting the cell as with STF treatment (FIG. 5B). To confirm that IRE1α was necessary for cell resistance to IR, we reproduced IR conditions on mouse embryonic fibroblasts (MEF) from IRE1α KO mice. Here also, we observe a clear decrease in cell survival compared to wild type cells (FIG. 5C).

The dichotomy between STF and siRNAs results prompted further investigation. In vivo immunofluorescence showed CIT-dependent CHOP upregulation, a factor also regulated by ATF6. We thus quantified CHOP mRNA levels: as in ATF6 experiments, CHOP mRNA was increased in cells subjected to IR; however treatment with STF significantly decreased CHOP expression to control levels (FIG. 5D). Knockdown of IRE1α reproduced this decrease; however the use of siRNA against XBP1 did not alter CHOP expression (FIG. 5E). Hence, IR-dependent upregulation of CHOP mRNA appears to be regulated by the endoribonuclease activity of IRE1α.

To ascertain that STF effects on CHOP expression were not due to indirect effects of the inhibitor, we explored whether STF impacted the expression of ATF6. Western blot analysis of ATF6 at different times of ischemia revealed a trend towards increased ATF6 expression after 12 h, but no significant differences at 24 h (data not shown). Hence, STF effect on CHOP expression appears to be independent of ATF6.

It thus appears that IRE1α is required for cell survival, but only if its RNase activity is inhibited. As a more in depth investigation dedicated to IRE1α would be beyond the scope of the present study, we oriented our work towards finalizing our understanding of the UPR involvement in IR.

Interplay Between the Pathways

We investigated possible redundancy between UPR pathways in cell protection against IR with different combinations of UPR modulators (data not shown). While combining STF and Salubrinal did not increase the viability of IR-subjected cells, AEBSF in conjunction with either STF or Salubrinal, or both, significantly increased the efficiency of the compounds in increasing cell survival.

Since AEBSF is a non-specific inhibitor of serine proteases, we attempted to confirm the specific role of ATF6 in this interplay using siRNA. While combining STF to siATF6 increased the protection provided by STF, there was no such additivity between siATF6 and Salubrinal, suggesting a non-specific action of AEBSF. Moreover, improvement of survival using AEBSF was unchanged by siATF6, confirming an important effect of the serine protease inhibitor independently of ATF6 (data not shown).

CHOP and GADD34 Expression are Altered During Kidney Transplantation in Patients

We analyzed a microarray database performed on human kidney grafts (GSE43974) to determine if markers of the UPR were altered according to the level of injury. Although there was no CIT data, we determined that there was an upregulation of CHOP mRNA in kidney grafts between time of procurement and end of preservation (data not shown). Moreover, R60 biopsies analysis revealed that GADD34, a target of ATF4, was downregulated in organs which would later develop delayed graft function (DGF) (data not shown).

Discussion

While constant advances in immunosuppressive therapeutics is successfully reducing the rate and severity of acute rejection episodes, long term graft and patient survival have not significantly been improved. This may be due to increasing ECD proportion, which could be linked to the high rate of DGF (from 25 to 50% in deceased donors), a post-transplant complication closely related to long term outcome. DGF occurrence and severity is linked to a number of parameters, among which IR and CIT. The correlation between CIT, IR, and long term outcome is now well defined. As CIT is used to estimate the intensity of cold ischemic lesions, we investigated the intracellular consequences of prolonged CIT, using both a preclinical model of kidney transplantation in the pig and an in vitro model of cold ischemia-reperfusion using endothelial cells. Indeed, in all vascularized organs the endothelium represents the interface between the graft and the host, playing a key role in the regulation of nutrient homeostasis and immune response. In cardiac IR, apoptosis of EC precedes cardiomyocytes' death, supporting their position as the first target of IR.

In vivo, we confirmed previous findings that the mitochondria are damaged in a time-dependent manner during cold storage, but we determined that another organelle was impacted: the endoplasmic reticulum (ER). While studies have shown that the ER is affected by warm IR, its involvement in the evolution of the lesion, particularly in the context of cold ischemia, remains unclear.

Each branch of the UPR has specific impact on the cell. IRE1α has both a kinase activity, permitting the assembly of the UPRosome, and an endoribonuclease (RNase) activity, responsible for the splicing of XBP1 mRNA but also other mRNAs and certain miRNAs. PERK is a transmembrane kinase that reprograms initiation factor eIF2a by phosphorylation, itself negatively regulated by the chaperone GADD34. Phosphorylated eIF2a decreases the ER load by reducing general translation and induces the translation of specific mRNAs, notably the one encoding ATF4. Finally, the ATF6 pathway is activated at the Golgi level after cleavage by site-1 and -2 proteases (S1P and S2P), releasing its N-terminal active portion. ATF6 notably regulates the ER chaperone GRP78.

We found that each arm of the UPR was differentially activated during IR, suggesting a finely tuned involvement in deciding cell fate:
the ATF6 pathway was activated late during cold ischemia, suggesting that its activation required a prolonged stress. However, ATF6 mRNA level remained unchanged during both ischemia and reperfusion suggesting a translational or post-translational regulation. We observed that inhibiting ATF6 increased EC survival. Furthermore, we demonstrated an additive effect between ATF6 inhibition, or knockdown, and other UPR modulators suggesting a decisive role for ATF6 in cell fate during IR. Indeed, it has been shown that ATF6 could trigger apoptosis in different settings. We determined that ATF6 could activate CHOP expression which appears to be CIT-dependent. In microarray of human kidney grafts, we also observed that CHOP was upregulated by cold storage.
regarding the PERK-eIF2α-ATF4 pathway, silencing either PERK or ATF4 did not alter cell survival, however maintaining the eIF2α-ATF4 axis active with Salubrinal improved cell survival, suggesting a protective role for this pathway only when pharmacologically overactivated. In human kidney grafts, GADD34, target of ATF4, was downregulated in organs which would develop DGF. Altogether, our data confirm a pro-survival role for the early activation of the eIF2α-ATF4 axis during IR, which may in part explain the cell's ability to withstand short periods of CIT.
the IRE1α-XBP1 pathway was the most complex. In line with recent findings in endothelial cell biology and angiogenesis, we found that silencing of either IRE1α or XBP1 (both XBP1s and XBP1u isoforms) decreased EC survival after IR. We inhibited IRE1α's RNase activity by STF083010 and 4μ8c (data not shown) and found that it increased EC survival in an IRE1α dependent manner. We determined a relationship between STF protection and the repression of CHOP expression, which strengthens the hypothesis that CHOP could be one of the main modulator of cell fate during cold IR. Involvement of CHOP in survival is further highlighted by our crosstalk studies in which combination of ATF6 inhibition to STF treatment, both decreasing CHOP expression, was highly protective against cell death. These results are in line with previous demonstration of strong interaction between the IRE1α-XBP1s and ATF6 pathways.

Decrease in CHOP expression was not reproduced by siXBP1, suggesting that CHOP is regulated independently of XBP1. This may be explained by the fact that IRE1α's RNase can cleave mRNAs and miRNAs other than XBP1; an activity named Regulated-IRE1α Dependent Degradation (RIDD). Indeed, the RIDD cleaves certain miRNAs targeting the pro-apoptotic caspase 2 or the thioredoxin-interacting protein, also involved in programmed cell death. In this regard, bioinformatic analyses using TarBase 6.0 revealed that CHOP mRNA is a predicted miRNA target of hsa-miR-96-5p which can be cleaved by IRE1α (Upton J-P, Wang L, Han D, Wang E S, Huskey N E, Lim L, et al. IRE1α Cleaves Select microRNAs During E R Stress to Derepress Translation of Proapoptotic Caspase-2. Science. 2012; 338:818-22.). Hence, inhibiting IRE1α's RNase activity may allow the stabilization of these miRNAs leading to inhibition of pro-apoptotic transcripts and improved cell survival. As no specific inhibitor of IRE1α's kinase activity is available, we cannot conclude on the role of this enzymatic activity on cell survival during IR.

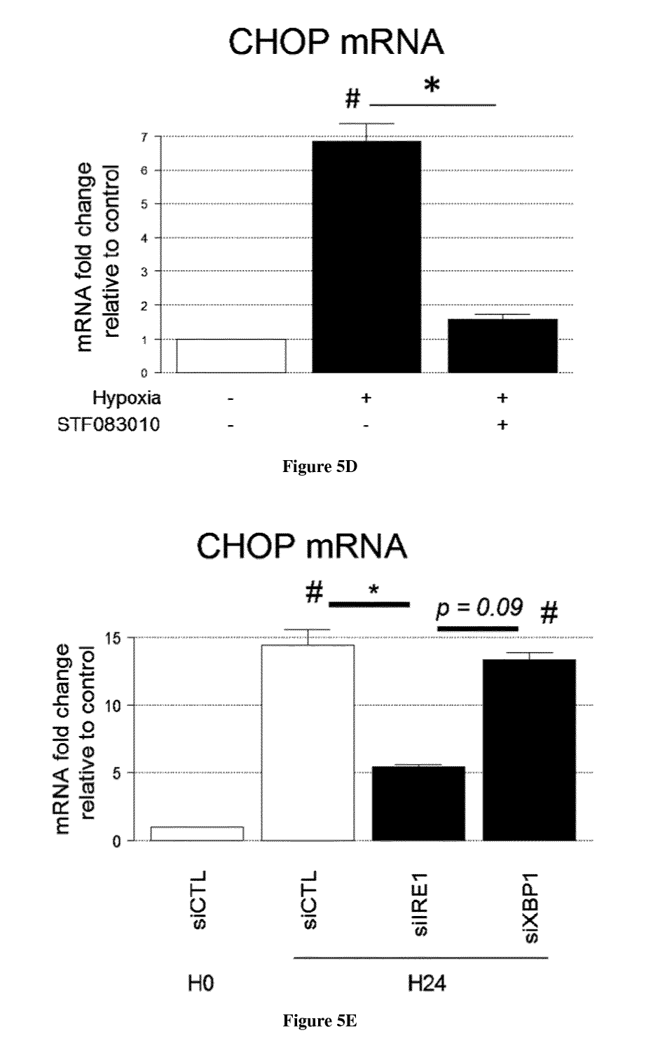

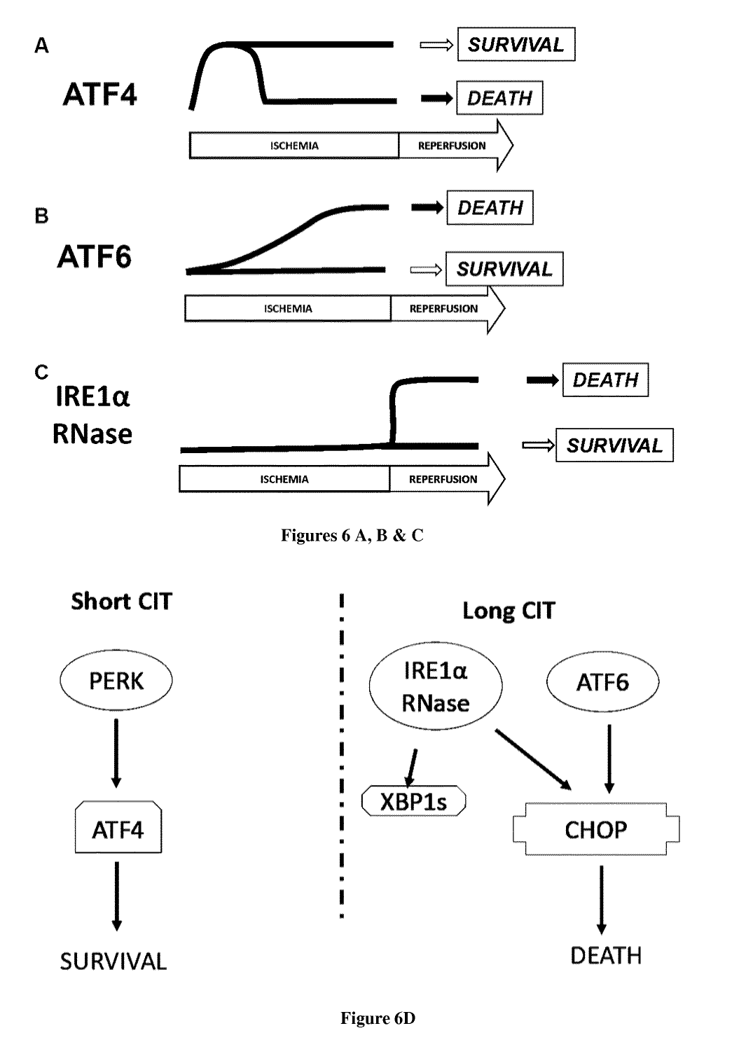

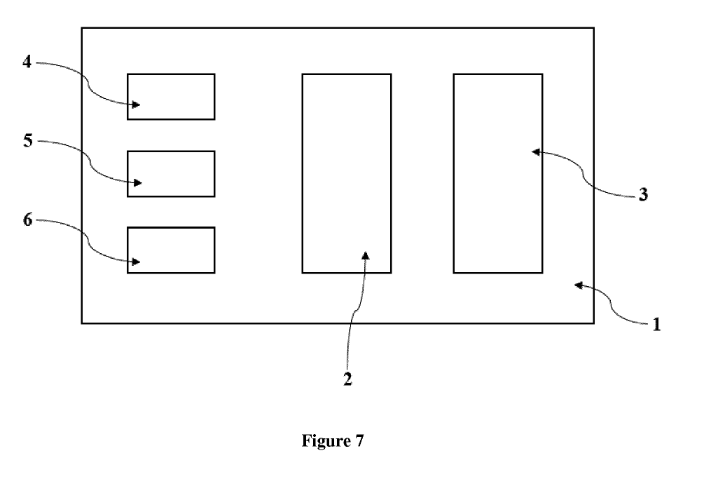

The UPR thus appears intricately modulated by CIT both in cells as well as in preclinical models and human kidney transplant patients, with specific kinetics for each pathways, linked to the level of injury (FIG. 6): -ATF4 (FIG. 6A) is only activated during the early phase of ischemia, at times corresponding to a minimal level of clinical complications, and if activation is maintained then survival is increased; -ATF6 (FIG. 6B) is activated later, at times generally associated with increased delayed graft function and long term complications; if inhibited, survival is increased; -IRE1α RNase activation (FIG. 6C) is not detected before reperfusion, however it is only observable when preceded by at least 12 hours of ischemia, hence also related to the level of injury; if it is inhibited, survival will increase. ATF6 and IRE1α RNase appear to kink to cell death through CHOP, in a CIT-dependent manner (FIG. 6D). This suggests that UPR mediators could represent critical therapeutic targets against IRI and markers of graft quality. Developing safe UPR-based therapies could be of great interest to reduce IRI in transplantation.

In conclusion, our results demonstrate that UPR could be a critical pathway underlying the relationship between CIT and graft outcome. Our data highlight the potential for UPR-based diagnostics to measure organ quality and therapeutics to enhance preservation quality, thereby improving transplantation efficiency of vascularized organs.

### REFERENCES

Throughout this application, various references describe the state of the art to which this invention pertains. The disclosures of these references are hereby incorporated by reference into the present disclosure.

1. A method of maintaining organ viability during cold ischemia comprising perfusing the organ with a preservation solution wherein:
an effective amount of an activator of PERK-ATF4 pathway is added in the first six hours of cold ischemia time;
and if the expected cold ischemia time exceeds 8 hours, adding to said preservation solution:
an effective amount of an inhibitor of the ATF6 pathway in the first twelve hours of cold ischemia time and/or,
an effective amount of an inhibitor of the RNase activity of IRE1α at least one hour before reperfusion of the organ.

2. The method according to claim 1, wherein the activator of PERK-ATF4 pathway is added immediately after cold ischemia starts.

3. The method according to claim 1, wherein the inhibitor of the ATF6 pathway is added in the first six hours of cold ischemia time.

4. The method according to claim 1, wherein the inhibitor of the ATF6 pathway is added in the first hour of cold ischemia time.

5. The method according to claim 1, wherein the activator of PERK-ATF4 pathway is an agent that inhibits the dephosphorylation of phosphorylated eIF2a.

6. The method according to claim 1, wherein the activator of PERK-ATF4 pathway is salubrinal.

7. The method according to claim 1, wherein the activator of PERK-ATF4 pathway is guanabenz.

8. The method according to claim 1, wherein the activator of PERK-ATF4 pathway is an inhibitor of GADD34 or PP1 gene expression, such as siRNA, antisense oligonucleotide or a ribozyme.

9. The method according to claim 1, wherein the inhibitor of the ATF6 pathway is an inhibitor of Site-1-protease or/and Site-2-protease.

10. The method according to claim 1, wherein the inhibitor of the ATF6 pathway is AEBSF.

11. The method according to claim 1, wherein the inhibitor of the ATF6 pathway is an inhibitor of ATF6 gene expression, such as siRNA, antisense oligonucleotide or a ribozyme.

12. The method according to claim 1, wherein the inhibitor of the RNase activity of IRE1α is STF083010.

13. A device for preserving an organ, said device comprising an organ container filled with a preservation solution, characterized in that said device further comprises one or more mean for injecting one or more compound into the organ container.

14. A device according to claim 13, wherein the injected compound is a therapeutic compound.

15. A device according to claim 13, wherein the injected compound is an activator of PERK-ATF4 pathway, an inhibitor of the ATF6 pathway or an inhibitor of the RNase activity of IRE1α.
